(19) United States
(12) Patent Application Publication
(10) Pub. No.: US 2020/0323436 A1
(43) Pub. Date: Oct. 15, 2020

(54) Heart Rate Display System

(71) Applicant: Stacey Knapp, Santa Cruz, CA (US)

(72) Inventor: Stacey Knapp, Santa Cruz, CA (US)

(21) Appl. No.: 16/865,436

(22) Filed: May 4, 2020

(51) Int. Cl. A61B 5/024; A61B 5/00

(52) U.S. Cl. CPC A61B 5/024; A61B 5/0022; A61B 5/744; A61B 5/7225; A61B 5/7246; A61B 5/681

(57) ABSTRACT

A heart rate display system is disclosed herein. The heart rate display system includes a smartphone device having a display device with at least one windowed area. The windowed area displays a rendering of a heart which encompasses greater than fifty percent of the at least one window area. The heart rate display system further includes an external heart rate monitor device communicatively paired to the smartphone device. The external heart rate monitor device continuously detects a user's heart rate. The heart rendering is a heart animation image representing the user's heart such that when the user's heart rate is at a baseline heart rate. The heart animation image represents the baseline heart rate and upon a change in the user's heart rate and the heart animation image changes to represent the change in the user's heart rate.

### CROSS REFERENCE TO RELATED APPLICATIONS

This patent application claims the benefit of U.S. Prov. Ser. No. 62/844,021 filed May 6, 2019 which is incorporated by reference herein.

### BACKGROUND

Trauma is defined as an overwhelming event that causes a perceived or real loss of safety by an individual. If adverse experiences occur in childhood, there is a higher risk that neurobiology will be impacted. This is especially true if the event occurs within the first years of life as these impacts may not be associated with a distinct memory, but rather from a subconscious reaction to perceived threats that manifest in what is commonly referred to as “fight or flight” symptoms. Indeed, this physiology of trauma may be imperceptible to the victim, or perceived as normal, and therefore difficult to recognize, diagnose and treat. The higher the number of Adverse Childhood Experiences (ACEs), the greater the likelihood that emotional dysregulation will have a lifelong impact on the individual. According the National Center for Disease Control (n.d), “almost two-thirds of adults surveyed reported at least one Adverse Childhood Experience (ACE)—and the majority of respondents that reported at least one ACE reported more than one.”

### BRIEF DESCRIPTION OF THE DRAWINGS

To facilitate understanding, identical reference numerals have been used, wherever possible, to designate identical elements that are common to the figures. The drawings are not to scale and the relative dimensions of various elements in the drawings are depicted schematically and not necessarily to scale. The techniques of the present invention may readily be understood by considering the following detailed description in conjunction with the accompanying drawings, in which:

FIG. 1 is an illustration of a smartphone device displaying a heart animation image within a window of a heart rate monitor and display software application.

FIG. 2 is an illustration of the heart animation image which changes color with the option to change size.

FIG. 3 is an illustration of the smartphone device which displays the result of a media player that streams an audio file with guided meditation content.

FIG. 4 is a heart rate display system in accordance with an embodiment of the present invention.

FIG. 5 is a method for displaying a user's heart rate in accordance with an embodiment of the present invention.

### DETAILED DESCRIPTION

Before the present invention is described in detail, it is to be understood that, unless otherwise indicated, this disclosure is not limited to specific procedures or articles, whether described or not.

It is further to be understood that the terminology used herein is for the purpose of describing particular embodiments only and is not intended to limit the scope of the present invention.

It must be noted that as used herein and in the claims, the singular forms “a,” and “the” include plural referents unless the context clearly dictates otherwise.

Where a range of values is provided, it is understood that each intervening value, to the tenth of the unit of the lower limit unless the context clearly dictates otherwise, between the upper and lower limit of that range, and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure. The term “about” generally refers to ±10% of a stated value.

A heart rate display system is disclosed herein. The heart rate display system includes a display device having at least one windowed area. The windowed area displays a rendering of a heart which encompasses at least fifty percent of the at least one window area. In some embodiments, the heart rendering is an animation representing a user's heart such that when the user's heart rate is at a baseline heart rate. The animation represents the baseline heart rate and upon a change in the user's heart rate, the animation changes to represent the change in the user's heart rate.

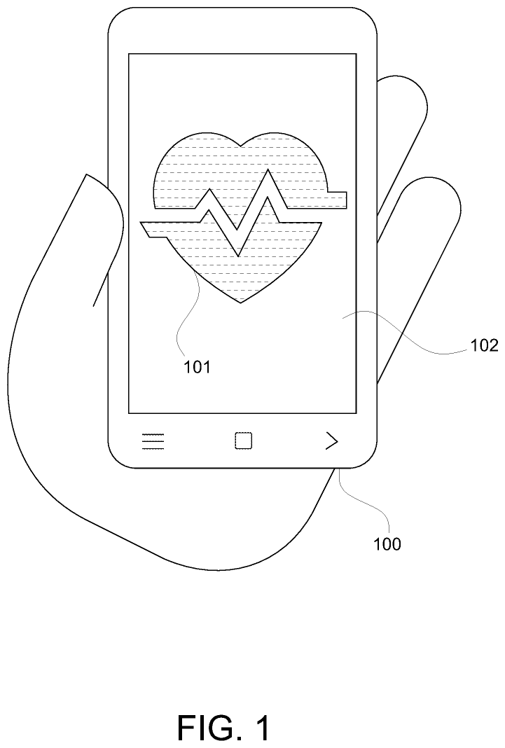

FIG. 1 is an illustration of a smartphone device 100 displaying a heart animation image 101 within a window 102 of a software application (not shown) and display software application. Accordingly, the smartphone device 100 hosts a software application (not shown) which can process heart monitoring data received from an external device (as will be discussed) and present said data in various manners. For instance, the heart rate animation is a manner in which the heart rate data is presented to a user within the window 102. In some implementations, the smartphone device 100 may be further equipped with a timer, and the hosted software application (not shown) may be further or alternatively equipped with a timer, for use while employing self-regulation strategies.

It should be understood by those having ordinary skill in the art that the present invention is not limited to a heart-shaped heart animation image 101 as shown in FIG. 1. Heart animation image 101 may be displayed in any manner that is consistent therewith the present invention.

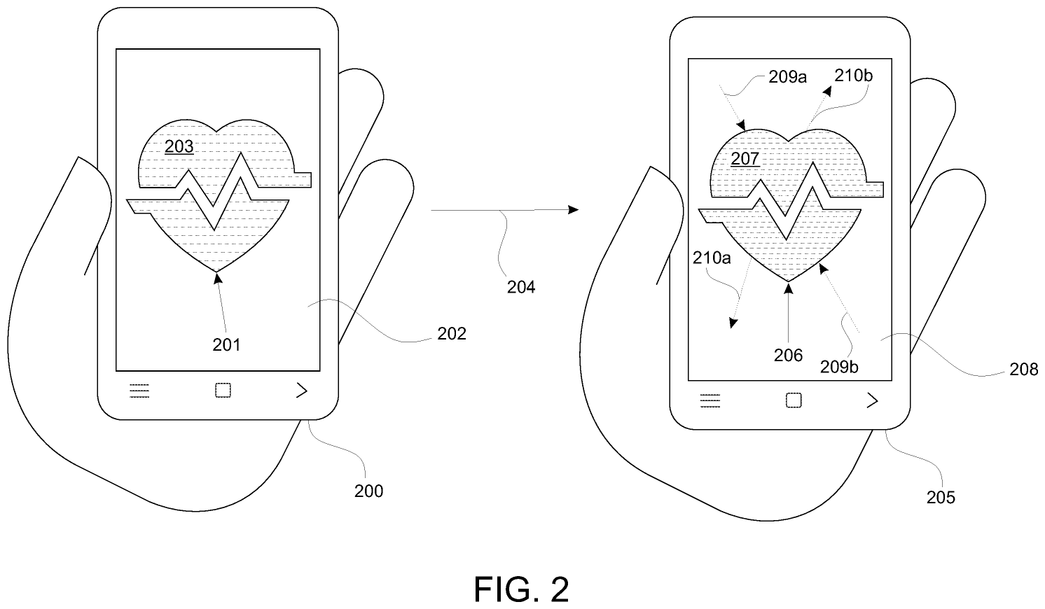

FIG. 2 is an illustration of the heart animation image which changes color with the option to change size. As shown, the smartphone device 200 displays a heart animation image 201 within a window 202 of a hosted software application (not shown). Notably, the heart rate animation image 201 may have a first visual effect (e.g., color 203). In some implementations, the color 203 displayed by the heart rate animation image 201 may be assigned to a baseline heart rate of a user.

In an event that a user's heart rate changes to a degree that is beyond a pre-determined threshold, the hosted software application may transition (e.g., see arrow 204) to a different color 204 as indicated by heart rate animation image 206.

In some implementations, a vivid blue-green indicates a baseline resting heart rate whereas heart rates that are higher than baseline will be indicated by purple, red, and orange. In addition, lower rates can be visualized with shade of green and blue. One having ordinary skill in the art will recognize that the aforementioned color schemes is exemplary and not dispositive.

In addition, the color changes may simulate the color transitions within a mood ring and thus may accomplish a “mood ring effect.” A mood ring effect is associated with the behavior of traditional mood rings. Traditional mood rings are attributed with including cholesteric liquid crystals which have the unique thermochromic material property. Most notably, cholesteric liquid crystals have the capability to change colors in the range of 0.1 to 0.2 seconds. As such, a software application that employs the present invention has the capability to change the color of the graphic visualizations in a swift fluid-like manner with forward and reversible capability.

Accordingly, the color changes in the graphic visualization can transition to colors in both a forward and reversible manner along a color spectrum. For example, when a user's heart rate increases, the heart animation rendering may transition in a forward manner to warmer colors (e.g., orange or red) on the color spectrum. Likewise, when a user's heart rate decrease, the heart animation rendering may transition in a reverse manner to cooler colors (e.g., green or blue) on the on the color spectrum.

Additionally, the heart rate animation image 206 displayed by the hosted software application (not shown) may change its size. Minimize arrows 209a, 209b and maximize arrows 210a, 210b of heart rate animation image 206 are illustrative that the heart rate animation image 206 can change in size. In some implementations, the change in size of the heart rate animation image 206 may be in response to a change in the user's heart rate.

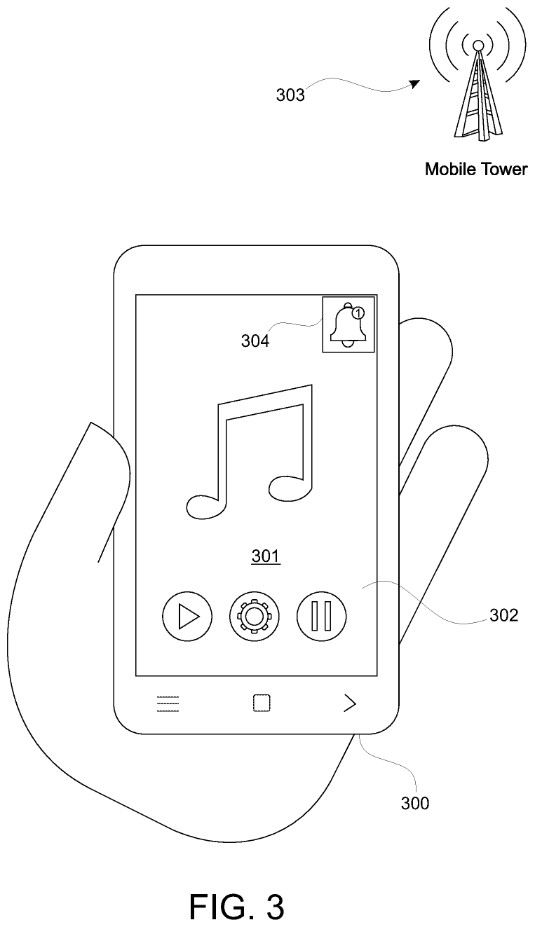

FIG. 3 is an illustration of the smartphone device 300 which displays the result of a media player 301 that streams an audio file (not shown) with guided meditation content. The smartphone device may receive information (e.g., streamed media) from a mobile tower 303. Notably, the guided meditation content may be used by users as a self-regulation strategy to get the user's heartrate back to a baseline condition. For example, the timer of the smartphone device 300 or the hosted software application (not shown) may play a Buddhist bowl chime audio track. In some implementations, while using other features, the heart visual will recede to an upper corner of the device.

In some implementations, the software application which employs the present invention may also display a bell icon 304 which can chime or make anu other suitable sound during the commission of the self-regulation strategies. For example, the bell icon 304 may be employed as a notification at the beginning or ending of a guided meditation audio file.

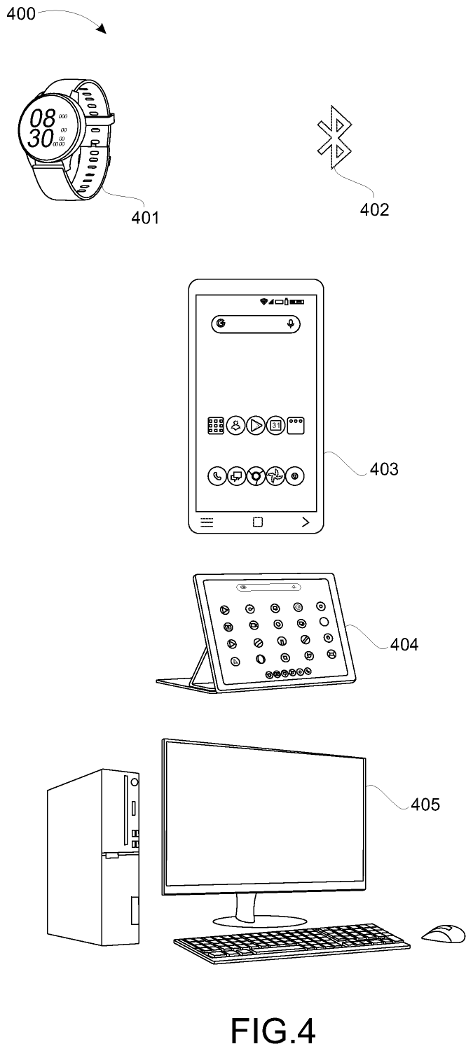

FIG. 4 is a heart rate display system 400 in accordance with an embodiment of the present invention. As shown, the heart rate display system 400 includes an external heart rate monitor device (e.g., a wrist monitor 401) paired (e.g., Bluetooth network 402) to an electronic communications device (e.g., smartphone device 403, tablet 404, and desktop computer 405). Accordingly, a wrist monitor 401 may be used to obtain a user's baseline heart rate which can be stored on an electronic communications device (e.g., smartphone device 403, tablet 404, and desktop computer 405).

The present invention is designed to support Trauma-Informed institutions and features individual passcode log in credentials and can accommodate multiple users on a software application which employs the present invention. Multiple users can be accommodated by associating each user with a 4-digit Personal Identification Number (PIN). Two user types can be specified-administrator and participant. Administrators can create new users (e.g., participants) and assign each new user a PIN. When a user logs in with their PIN, the users can access only the data associated with their account.

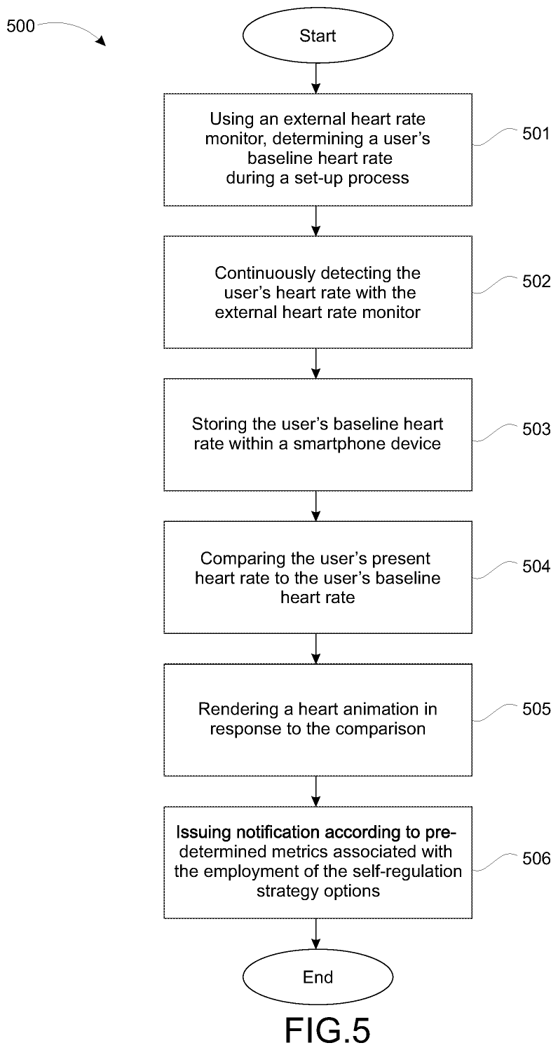

FIG. 5 is a method 500 for displaying a user's heart rate in accordance with an embodiment of the present invention. Method 500 begins with using an external heart rate monitor device, determining a user's baseline heart rate during a set-up process (block 501). Next, method 500 include continuously detecting the user's heart rate with the external heart rate monitor device (block 502). Additionally, method 500 includes storing the user's baseline heart rate within a smartphone device. In some embodiments, text-based communication mechanism of the smartphone device sends a text message to an emergency contact phone list when the comparison yields a change to the user's baseline heart rate that is above a pre-determined threshold.

Furthermore, method 500 includes comparing the user's present heart rate to the user's baseline heart rate (block 504). In some embodiments, when the comparison yields a change to the user's baseline heart rate that is above a pre-determined threshold, the smartphone device provides self-regulation strategy options to the user. The self-regulation strategy options may include at least one audio or video file with guided meditation media. In some embodiments, the guided meditation media includes movement-based-self regulation strategies and postural restoration breathing strategies. The self-regulation strategy options are streamed from an external service to the smartphone device.

Further, method 500 includes rendering a heart animation image in response to the comparison (block 505). The hearing animation may appear as the heart animation image shown in FIGS. 1 and 2.

Lastly, the method 500 includes issuing notification according to pre-determined metrics associated with the employment of the self-regulation strategy options (block 506).The timer of the software application that employs the present invention can be used during self-regulation strategies that indicates time by, for example, a Buddhist bowl chime audio track. In some implementations, while using other features, the heart visual will recede to an upper corner of the device.

While illustrative implementations of the application have been described in detail herein, it is to be understood that the inventive concepts may be otherwise variously embodied and employed, and that the appended claims are intended to be construed to include such variations, except as limited by the prior art.

Reference throughout this specification to “one implementation” or “an implementation” means that a particular feature, structure, or characteristic described in connection with the implementation is included in at least one implementation of the present invention. Thus, the appearances of the phrases “in one implementation” or “in some implementations” in various places throughout this specification are not necessarily all referring to the same implementation. Furthermore, the particular features, structures, or characteristics may be combined in any suitable manner in one or more implementations.

The present invention can be used by educators, educational staff, students, parents or anyone interested in working on decreasing the physiological symptoms associated with stress and/or Adverse Childhood Experiences (ACEs). Educators also benefit from the practice of self-care and mindfulness strategies and will be encouraged to model these strategies in classrooms. Students working with one-on-one mentors, behavioral staff and/or school nurses can benefit from the repetitive practice offered in intervention settings of using the device to measure and compare heart rates before and after strategy practice.

Systems and methods describing the present invention have been described. It will be understood that the descriptions of some embodiments of the present invention do not limit the various alternative, modified, and equivalent embodiments which may be included within the spirit and scope of the present invention as defined by the appended claims. Furthermore, in the detailed description above, numerous specific details are set forth to provide an understanding of various embodiments of the present invention. However, some embodiments of the present invention may be practiced without these specific details. In other instances, well known methods, procedures, and components have not been described in detail so as not to unnecessarily obscure aspects of the present embodiments.

What is claimed is:

1. A system comprising:
a display device having at least one windowed area;
the at least one windowed area displays a rendering of a heart which encompasses at least fifty percent of the at least one window area;
wherein the heart rendering is a an animation representing a user's heart such that when the user's heart rate is at a baseline heart rate, the animation represents the baseline heart rate and upon a change in the user's heart rate, the animation changes to represent the change in the user's heart rate.

2. The system of claim 1, wherein the display device is a component of at least one of a smartphone, computing tablet, kiosk, desktop computer, or any other computing device with a user interface.

3. The system of claim 2, further comprising an external heart rate monitor device communicatively paired to the computing device which has the display device.

4. The system of claim 3, wherein the external heart rate monitor device is a wristband heart rate monitor.

5. The system of claim 1, wherein when it is detected that the user's heart rate exceeds the baseline heart rate, the display device triggers a first change to the animation and when the heart rate is lower than the baseline heart rate, the display device triggers a second change to the animation.

6. The system of claim 5, wherein the first change to the animation is a first change in color and the second change to the animation is a second change in color.

7. The system of claim 5, wherein the first change in color and the second change in color occur as a transition in color shade similar to color transitions in a mood ring.

8. The system of claim 5, further comprising a first smartphone device which hosts a first user and a second smartphone device hosts a second smartphone device.

9. A heart rate display system, comprising:
a smartphone device having a display device with at least one windowed area;
the windowed area displays a rendering of a heart which encompasses greater than fifty percent of the at least one window area;
an external heart rate monitor device communicatively paired to the smartphone device, the external heart rate monitor device continuously detects a user's heart rate;
wherein the heart rendering is a heart animation image representing the user's heart such that when the user's heart rate is at a baseline heart rate, the heart animation image represents the baseline heart rate and upon a change in the user's heart rate, the heart animation image changes to represent the change in the user's heart rate.

10. The system of claim 9, wherein the at least one windowed area consumes the entire display area.

11. The system of claim 9, wherein when the heart rate is different from the baseline heart rate, meditation content retrieved by the smartphone device is made available to the user.

12. The system of claim 9, wherein the external heart rate monitor device is used to determine the user's baseline heart rate during a set-up process which provides a timed measurement activity which offers tips for obtaining best measurements for the baseline heart rate.

13. The system of claim 9, wherein the change in the user's heart rate is detected using a differencing and averaging algorithm.

14. The system of claim 13, wherein the differencing and averaging algorithm is the Karmarkar-Karp differencing algorithm.

15. A method, comprising:
using an external heart rate monitor device, determining a user's baseline heart rate during a set-up process;
storing the user's baseline heart rate within a smartphone device;
continuously detecting the user's heart rate with the external heart rate monitor device;
comparing the user's present heart rate to the user's baseline heart rate; and
rendering a heart animation image rendering in response to the comparison;
wherein the heart animation image rendering represents the baseline heart rate and upon a change in the user's heart rate, the heart animation image rendering changes to represent the change in the user's heart rate.

16. The method of claim 15, wherein text-based communication mechanism of the smartphone device sends a text message to an emergency contact phone list when the comparison yields a change to the user's baseline heart rate that is above a pre-determined threshold.

17. The method of claim 15, wherein when the comparison yields a change to the user's baseline heart rate that is above a pre-determined threshold, the smartphone device provides self-regulation strategy options to the user.

18. The method of claim 17, wherein the self-regulation strategy options include at least one audio or video file with guided meditation media.

19. The method of claim 17, wherein the self-regulation strategy options are streamed from an external service to the smartphone device.

20. The method of claim 19, further comprising issuing notification according to pre-determined metrics associated with the employment of the self-regulation strategy options.
